SEQUENCE LISTING (5 sequences)

SEQ ID NO: 1 - DNA, 13 bp, Saccharomyces cerevisiae
tgcgattgaatgg

SEQ ID NO: 2 - DNA, 11 bp, Saccharomyces cerevisiae
aagattcgatg

SEQ ID NO: 3 - DNA, 11 bp, Saccharomyces cerevisiae
tcccaaacaac

SEQ ID NO: 4 - DNA, 13 bp, Saccharomyces cerevisiae
accgattgaatgg

SEQ ID NO: 5 - DNA, 13 bp, Saccharomyces cerevisiae
tatcccaaacaac

(19) United States
(12) Patent Application Publication
(10) Pub. No.: US 2015/0292039 A1
(43) Pub. Date: Oct. 15, 2015

(54) METHOD TO AMPLIFY NUCLEIC ACIDS OF FUNGI TO GENERATE FLUORESCENCE LABELED FRAGMENTS OF CONSERVED AND ARBITRARY PRODUCTS

(71) Applicant: E I DU PONT DE NEMOURS AND COMPANY, Wilmington, DE (US)

(72) Inventors: Xuan Peng, Hockessin, DE (US); Mark A. Jensen, West Chester, PA (US)

(21) Appl. No.: 14/680,480

(22) Filed: Apr. 7, 2015

Related U.S. Application Data
(60) Provisional application No. 61/977,878, filed on Apr. 10, 2014.

(51) Int. Cl. C12Q 1/68

(52) U.S. Cl. CPC C12Q 1/6895; C12Q 2600/16

(57) ABSTRACT

Disclosed herein are methods for the identification of the species, serotype, and strain of a fungi. Also disclosed are primers for use in detecting such fungi and kits comprising such primers.

### CROSS-REFERENCE TO RELATED APPLICATION

This application claims priority from U.S. Provisional Application Ser. No. 61/977,878 filed on Apr. 10, 2014, which is hereby incorporated by reference in its entirety.

### FIELD OF INVENTION

The field relates to methods of fungi identification using random amplified polymorphic DNA (RAPD) PCR.

### BACKGROUND OF INVENTION

Fungi, with over 1,500,000 species and genome size of 10-100 mega bases, make up the most diverse kingdom of the tree of life. Fungi are characterized by the absence of chlorophyll and the presence of a rigid cell wall composed of chitin, mannans, and sometimes cellulose. Fungus (fungi) include but are not limited to mushrooms, yeasts, rusts, molds, and smuts. (http://www.biology-online.org/dictionary/Fungus).

Fungi can become pathogens as many fungi produce mycotoxins that are toxic to humans and animals. Fungi can also cause severe crop losses and lead to food spoilage. This can have a major impact on food supplies and local economies. Pathogenic fungal contamination is of public health concern. Of the 45 recalls by the US Food and Drug Administration (FDA), 16% were due to mold or yeast in 2012, of which the multistate outbreak of fungal meningitis that claimed 48 lives was caused by product contamination by a common mold Exserohilum rosstrtum in methylprednisolone acetate injections. The early, rapid, and accurate identification of the pathogenic fungi is therefore important for both the public health agencies and hospital epidemiologists to pin point the link between patient and the origin of the outbreak and come up with timely and appropriate management. Manufacturers of food and feed, pharmaceutical and personal care products also need this information to enhance the safety of their products and protect their brands.

Central to the field of microbiology is the ability to positively identify microorganisms at the level of genus, species, or serotype. Correct identification is not only an essential tool in the laboratory, but it plays a significant role in the control of fungal contamination in the processing of foods, beverages, pharmaceutical and personal care products, as well as the production of agricultural products, and the monitoring of environmental media, such as ground water and air. Typically, pathogen identification has relied on methods for distinguishing phenotypic aspects, such as growth or motility characteristics, and for immunological and serological characteristics. Selective growth procedures and immunological methods are the traditional methods of choice for fungal identification and these can be effective for the presumptive detection of a large number of species within a particular genus. However, these methods are time consuming and are subject to error. Selective growth methods require culturing and subculturing in selective media, followed by subjective analysis by an experienced investigator. Immunological detection (e.g., ELISA) is more rapid and specific, however, it still requires growth of a significant population of organisms and isolation of the relevant antigens. Therefore the identification of fungi, especially filamentous fungi, has historically been a very difficult task due to inadequate comprehension of the whole fungal speciation connected with population biology, ecology, evolution and phylogeny. The morphological and physiological based standard biological methods are insufficient in many cases. They require several days or even weeks and often tend to fail. For these reasons, interest has turned to detection of fungi based on nucleic acid sequence.

The DNA assays are substantially more accurate and reproducible than morphological and physiological based phenotypic methods. This is also well understood and accepted by U.S. Food and Drug Administration as stated in the “FDA Guidance for Industry. Sterile Drug Products Produced by Aseptic Processing—Current Good Manufacturing Practice” early in 2004. Nucleic acid polymorphism provides a means to identify species, serotypes, strains, varieties, breeds, or individuals based on differences in their genetic make up. Nucleic acid polymorphism can be caused by nucleotide substitution, insertion, or deletion. The ability to determine genetic polymorphism has widespread application in areas such as genome mapping, genetic linkage studies, medical diagnosis, epidemiological studies, forensics, and agriculture.

Currently available methods to identify and characterize fungi are either labor-intensive and time-consuming, or require the user to know the genus of the fungi in advance. No platform is capable of doing strain identification and sub-species tracking simultaneously, or close to an ideal method which should be rapid, robust, produce objective data, differentiate all epidemiologically unrelated strains and group together all same source derived isolates.

The experimental approach of using short, conserved ribosomal primers to generate both conserved rDNA fragments and arbitrary amplification products is presented in U.S. Pat. No. 5,753,467. Microbial identification at the level of genus and species is accomplished by the characterization of variations in length and number of fragments located between highly conserved rDNA sequences. The level of identification is extended to the level of serotype and strain by the concurrent amplification of additional arbitrary regions of the microbial genome. These arbitrary amplification events are referred to as Random Amplified Polymorphic DNA (RAPD). This approach has not previously been applied to fungi.

The rDNA genetic locus is a genetic unit, which is found in fungi cells. The conserved amplification targets are those sequences found in the spacer region between the 18S, 5.8S, and 28S genes which code for ribosomal DNA (rDNA). These targets are amplified from conserved sequences in the adjacent 18S, 5.8S, and 28S regions. Significant portions of the nucleic acid sequence, which make up this genetic locus, are common to all fungi (FIG. 1 shows a generalized schematic of this locus). The overall relatedness of the 18S, 5.8S, and 28S regions of this genetic locus has been used as a tool to classify differing species of fungi.

The approach described in U.S. Pat. No. 5,753,467 makes use of short primers of 10-12 bases in length. The products generated by these primers are separated through the use of an electrophoretic separation in either agarose or polyacrylamide. The fragments are then visualized through staining with ethidium bromide. During the gel loading process, the PCR products could potentially contaminate the laboratory environment.

### SUMMARY OF INVENTION

One aspect is for a method for the identification of the species, serotype, and strain of a fungi comprising: (a) amplifying DNA comprising variable sequences interspersed between highly conserved rDNA sequences by PCR and amplifying additional genomic sequences by random amplified polymorphic DNA (RAPD) PCR using a first primer of 13 bases in length and a second primer of 11-13 bases in length, said first primer comprising: (i) at least 11 contiguous bases from a highly conserved 18S rDNA region and (b) separating the amplified DNA produced in step (a).

Another aspect is for an isolated polynucleotide selected from the group consisting of SEQ ID NO:1, SEQ ID NO:2, SEQ ID NO:3, SEQ ID NO:4, SEQ ID NO:5, and the full-length complements thereof.

A further aspect is for a kit comprising a set of primers comprising at least one of PCR primers SEQ ID NO:1 and SEQ 1D NO:4 and at least one of SEQ ID NO:2, SEQ ID NO:3, and SEQ ID NO:5, wherein SEQ ID NO:1 and SEQ ID NO:5 are labeled with a fluorophore.

Other advantages will become apparent to those skilled in the art upon reference to the detailed description that hereinafter follows.

### BRIEF DESCRIPTION OF THE FIGURES

FIG. 1 is a schematic of the ribosomal cassette of repetitive genes in fungi. The 5.8S gene is flanked by the 18S and 28S genes. ETS: External Transcribed Spacer. ITS: Internal Transcribed Spacer.

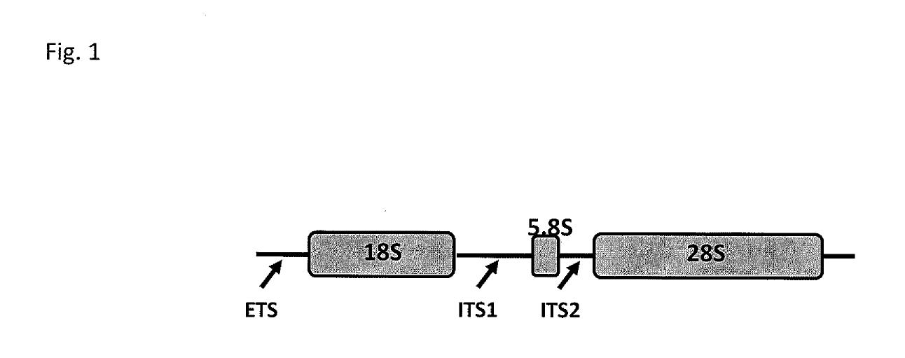

FIG. 2 shows electropherograms of PCR products generated with 6-FAM labeled Q_{—}18S-660-TG11F (SEQ ID NO:1) and unlabeled M_{—}5.8S-rc1084-11R (SEQ ID NO:2) primer group for Penicillium chrysogenum (Pchr), and Saccharomyces cerevisiae (Scer).

FIG. 3 shows electropherograms of PCR products generated with 6-FAM labeled Q_{—}18S-660-TG11F (SEQ ID NO:1) and unlabeled M_{—}5.8S-rc1084-11R (SEQ ID NO:2) primer group for Aspergillus niger (Anig), Candida albicans (Calb) and Candida tropicalis (Ctro).

FIG. 4 shows electropherograms of PCR products generated with 6-FAM labeled Q_{—}18S-660-TG11F (SEQ ID NO:1) and unlabeled M_{—}5.8S-rc1084-11R (SEQ ID NO:2) primer group for Cryptococcus neoformans (Cneo), Fusarium solani (Fsol), Rhodotorula mucilaginosa (Rmuc) and Zygosaccharomyces rouxii (Zrou)

### BRIEF DESCRIPTION OF THE BIOLOGICAL SEQUENCES

The following sequences comply with 37 C.F.R. §§1.821-1.825 (“Requirements for Patent Applications Containing Nucleotide Sequences and/or Amino Acid Sequence Disclosures—the Sequence Rules”) and are consistent with World Intellectual Property Organization (WIPO) Standard ST.25 (1998) and the sequence listing requirements of the European Patent Convention (EPC) and the Patent Cooperation Treaty (PCT) Rules 5.2 and 49.5(a-bis), and Section 208 and Annex C of the Administrative Instructions. The symbols and format used for nucleotide and amino acid sequence data comply with the rules set forth in 37 C.F.R. §1.822.

SEQ ID NO:1 is a forward primer containing a 13 bp sequence comprised of 11 bp from 18S rDNA and a 2 base mismatch on the 5′ end.

SEQ ID NO:2 is a reverse primer containing an 11 bp sequence from 5.8S rDNA.

SEQ ID NO:3 is a reverse primer containing an 11 bp sequence from 28S rDNA.

SEQ ID NO:4 is a forward primer containing a 13 bp sequence from 18S rDNA.

SEQ ID NO:5 is a reverse primer containing a 13 bp sequence comprised of 11 bp from 28S rDNA and a 2 base mismatch on the 5′ end.

### DETAILED DESCRIPTION OF PREFERRED EMBODIMENTS

Applicants specifically incorporate the entire contents of all cited references in this disclosure. Further, when an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the invention be limited to the specific values recited when defining a range.

In this disclosure, a number of terms and abbreviations are used. The following definitions apply unless specifically stated otherwise.

As used herein, the articles “a”, “an”, and “the” preceding an element or component of the invention are intended to be nonrestrictive regarding the number of instances (i.e., occurrences) of the element or component. Therefore “a”, “an” and “the” should be read to include one or at least one, and the singular word form of the element or component also includes the plural unless the number is obviously meant to be singular.

The term “comprising” means the presence of the stated features, integers, steps, or components as referred to in the claims, but does not preclude the presence or addition of one or more other features, integers, steps, components or groups thereof. The term “comprising” is intended to include embodiments encompassed by the terms “consisting essentially of” and “consisting of”. Similarly, the term “consisting essentially of” is intended to include embodiments encompassed by the term “consisting of”.

As used herein, the term “about” modifying the quantity of an ingredient or reactant employed refers to variation in the numerical quantity that can occur, for example, through typical measuring and liquid handling procedures used for making concentrates or use solutions in the real world; through inadvertent error in these procedures; through differences in the manufacture, source, or purity of the ingredients employed to make the compositions or carry out the methods; and the like. The term “about” also encompasses amounts that differ due to different equilibrium conditions for a composition resulting from a particular initial mixture. Whether or not modified by the term “about”, the claims include equivalents to the quantities.

Where present, all ranges are inclusive and combinable. For example, when a range of “1 to 5” is recited, the recited range should be construed as including ranges “1 to 4”, “1 to 3”, “1-2”, “1-2 & 4-5”, “1-3 & 5”, and the like.

The term “oligonucleotide” as used herein refers to a molecule comprised of two or more deoxyribonucleotides or ribonucleotides.

The terms “amplification” or “amplify” as used herein include methods for copying a target nucleic acid, thereby increasing the number of copies of a selected nucleic acid sequence. Amplification may be exponential or linear. A target nucleic acid may be either DNA or RNA. The sequences amplified in this manner form an “amplicon”.

The term “nucleic acid” refers to a polymer of ribonucleic acids or deoxyribonucleic acids, including RNA, mRNA, rRNA, tRNA, small nuclear RNAs, cDNA, DNA, PNA, RNA/DNA copolymers, or analogues thereof. Nucleic acids may be obtained from a cellular extract, genomic (gDNA) or extragenomic DNA, viral RNA or DNA, or artificially/chemically synthesized molecules.

The term “complementary” refers to nucleic acid sequences capable of base-pairing according to the standard Watson-Crick complementary rules, or being capable of hybridizing to a particular nucleic acid segment under relatively stringent conditions. Nucleic acid polymers are optionally complementary across only portions of their entire sequences.

The term “fungi” refers to eukaryotic organisms (each containing a membrane-bound nucleus) that develop from reproductive bodies called spores.

The term “mold” refers to a fungus that grows in the form of multicellular filaments called hyphae.

The term “target”, “target sequence”, or “target nucleotide sequence” refers to a specific nucleic acid sequence, the presence, absence or abundance of which is to be determined.

As used herein, a “primer” for amplification is an oligonucleotide that is complementary to a target nucleotide sequence and leads to addition of nucleotides to the 3′ end of the primer in the presence of a DNA or RNA polymerase. The 3′ nucleotide of the primer should generally be identical to the target sequence at a corresponding nucleotide position for optimal expression and amplification. The term “primer” as used herein includes all forms of primers that may be synthesized including peptide nucleic acid primers, locked nucleic acid primers, phosphorothioate modified primers, labeled primers, and the like. As used herein, a “forward primer” is a primer that is complementary to the anti-sense strand of dsDNA. A “reverse primer” is complementary to the sense-strand of dsDNA. Primers are typically between about 10 and about 100 nucleotides in length, preferably between about 15 and about 60 nucleotides in length, and most preferably between about 20 and about 30 nucleotides in length.

An oligonucleotide (e.g., a probe or a primer) that is specific for a target nucleic acid will “hybridize” to the target nucleic acid under suitable conditions. As used herein, “hybridization” or “hybridizing” refers to the process by which an oligonucleotide single strand anneals with a complementary strand through base pairing under defined hybridization conditions. “Specific hybridization” is an indication that two nucleic acid sequences share a high degree of complementarity. Specific hybridization complexes form under permissive annealing conditions and remain hybridized after any subsequent washing steps. Permissive conditions for annealing of nucleic acid sequences are routinely determinable by one of ordinary skill in the art and may occur, for example, at 65° C. in the presence of about 6×SSC. Stringency of hybridization may be expressed, in part, with reference to the temperature under which the wash steps are carried out. Such temperatures are typically selected to be about 5° C. to about 20° C. lower than the thermal melting point (Tm) for the specific sequence at a defined ionic strength and pH. One set of preferred conditions uses a series of washes starting with 6×SSC, 0.5% SDS at room temperature for 15 min, then repeated with 2×SSC, 0.5% SDS at 45° C. for 30 min, and then repeated twice with 0.2×SSC, 0.5% SDS at 50° C. for 30 min. A more preferred set of conditions uses higher temperatures in which the washes are identical to those above except for the temperature of the final two 30 min washes in 0.2×SSC, 0.5% SDS was increased to 60° C. Another preferred set of stringent hybridization conditions is 0.1×SSC, 0.1% SDS, 65° C. and washed with 2×SSC, 0.1% SDS followed by a final wash of 0.1 ×SSC, 0.1% SDS, 65° C.

The term “label” refers to any detectable moiety. A label may be used to distinguish a particular nucleic acid from others that are unlabeled, or labeled differently, or the label may be used to enhance detection.

The term “specimen” means a biological sample such as saliva, stools, urine, blood, gastric biopsy, gastrointestinal tissue, tumor cells, mucus secretions, dental plaque, and other biological tissues; meat products; food products; and environmental samples such as soil or water.

The term “yeast” refers to any of various small, single-celled fungi of the phylum Ascomycota that reproduce by fission or budding, the daughter cells often remaining attached, and that are capable of fermenting carbohydrates into alcohol and carbon dioxide.

### Nucleic Acid Detection

To avoid the potential hazard of laboratory environment contamination, the present method requires the use of fluorescence labeled primers. Products generated from these primers can be directly detected by capillary electrophoresis. Use of short fluorescence labeled primers, 10 to 12 bases, presents a difficulty because the presence of the fluorescent moiety makes such primers a poor substrate for DNA polymerases. Longer primers with 100% homology to the conserved sequences cannot be substituted because such primers will amplify only the ribosomal fragments without the arbitrarily primed pattern elements that are critical to strain level differentiation.

The minimum length required for incorporation of a fluorescence labeled primer was 13 bases. Since 13-base primers with a perfect match to the ribosomal site amplified only the ribosomal fragments, it was necessary to employ an at least 2-base mismatch on the 5′ end of the fluorescence labeled primer. Since only the last 11 bases matched the ribosomal sequence, such primers are capable of amplifying both ribosomal fragments and arbitrary genomic fragments simultaneously.

More particularly, the present method comprises amplifying DNA comprising variable sequences interspersed between highly conserved rDNA sequences by PCR and amplifying additional genomic sequences by RAPD PCR using a first primer of at least 13 bases in length and a second primer of 11-13 bases in length. The first primer comprises at least 11 contiguous bases from a highly conserved 18S rDNA region and can further include a fluorescent label. The second primer comprises at least 11 contiguous bases from a highly conserved 5.8S rDNA or a highly conserved 28S rDNA and can further include a fluorescent label. When the first primer includes a fluorescent label then the second primer does not include a fluorescent label and when the first primer does not include a fluorescent label then the second primer does include a fluorescent label. Thus the fluorescent label may be included in either the first primer or the second primer. In a second step, the method comprises separating the amplified DNA produced in the amplifying step.

The method described herein is useful in identifying a wide variety of fungi, including yeasts and molds. Representative but not exhaustive of the many types of organisms including both genus, species and serotype that may be elicited through the use of the present procedures are Acremonium, Alternaria, Amylomyces, Anthrodema, Aspergillus, Aureobassidium, Auxarthron, Blastoshizomyces, Botrytis, Brettanomyces (Dekkera), Byssochlamys, Candida, Cladosporium, Corynascus, Cryptococcus, Debaryomyces, Dekkera, Dictostelium, Emericella, Eupenicillium, Eurotium, Fonsecaea, Fusarium, Geomyces, Geosmithia, Geotrichum, Hyphopichia, Kloeckera, Kluyveromyces, Malbranchea, Monascus, Mucor, Myceliophthora, Neosartorya, Neurospora, Paecilomyces, Penicillium, Phoma, Pichia (Hansenula), Pilaira, Rasamsonia, Remersonia, Rhizomucor, Rhizopus, Rhodotorula, Saccaromyces, Scopulariopsis, Sporobolomyces, Sporothrix, Stachybotrys, Taifanglania, Talaromyces, Thermoascus, Thielavia, Torulaspora (Debaryomyces), Trichoderma, Trichosporon, Trichothecium, Ulocladium, Ustilago, Verticillium, Wallemia, Yarrow, and Zycgosacharomyces. Such a listing may form a database of previously visualized products which when compared to the electrophoresed, visualized fragment products according to the present method, afford an identification of the species (and the serotype and strain if applicable).

It is readily appreciated by one skilled in the art that the present method may be applied to fungi in the context of a wide variety of circumstances. Thus, a preferred use of the present invention is in the identification of fungi in foods, beverages, pharmaceutical and personal care products etc. Additionally, research directed to fungal infections in humans, other animals, and plants would benefit from the procedure herein.

Nucleic acids may be isolated from a sample according to any methods well known to those of skill in the art. If necessary the sample may be collected or concentrated by centrifugation and the like. The cells of the sample may be subjected to lysis, such as by treatments with enzymes, heat, surfactants, ultrasonication, or combination thereof.

Various methods of nucleic acid extraction are suitable for isolating nucleic acids. Suitable methods include phenol and chloroform extraction. See, e.g., Charles S. Hoffman, Current Protocols in Molecular Biology, John Wiley & Sons, Inc. Press (1997).

RAPD PCR is disclosed in U.S. Pat. No. 5,126,239 (see also, Williams et al., Nucleic Acids Res. 18:6531-34 (1990)). The approach describes the use of a small oligonucleotide, i.e., greater than seven nucleotides, of arbitrary composition in a DNA amplification reaction. Short primers are used in order that complementary and reverse complementary sequences to the primer can be found at distances along the genome which are sufficiently small that DNA amplification can take place. The fragments generated in the amplification process are called RAPD markers. These RAPD markers show a size distribution which is sensitive to modest differences in the genomic makeup of the DNA used in the amplification process.

As noted in U.S. Pat. No. 5,753,467, the process of U.S. Pat. No. 5,126,239 requires 45 cycles, which frequently results in the formation of secondary amplification products and nonspecific DNA synthesis. A product profile background which contains high levels of such secondary amplification products and nonspecific DNA can severely restrict the ability of pattern recognition software to compare such a product profile with a known database. The process disclosed herein, however, uses fewer amplification cycles with longer annealing times to produce a far less complex product profile with a significantly reduced nonspecific DNA background.

The skilled artisan is capable of designing and preparing arbitrary primers that are appropriate for RAPD PCR. The length of the amplification primers depends on several factors including the nucleotide sequence identity and the temperature at which these nucleic acids are hybridized or used during in vitro nucleic acid amplification. The considerations necessary to determine a preferred length for an amplification primer of a particular sequence identity are well known to the person of ordinary skill in the art.

Primers that amplify a nucleic acid molecule can be designed using, for example, a computer program such as OLIGO (Molecular Biology Insights, Inc., Cascade, Colo.). Important features when designing oligonucleotides to be used as amplification primers include, but are not limited to, an appropriate size amplification product to facilitate detection (e.g., by electrophoresis), similar melting temperatures for the members of a pair of primers, and the length of each primer (i.e., the primers need to be long enough to anneal with sequence-specificity and to initiate synthesis but not so long that fidelity is reduced during oligonucleotide synthesis). Preferred primers, along with their targets, are described in Table 1 below.

As discussed in U.S. Pat. No. 5,753,467, a significant degree of intramolecular hybridization is known to occur within the rDNA genetic locus. The resulting secondary structure frequently makes it difficult for amplification primers to compete for hybridization sites. In order to enhance the amplification of fragments contained within the rDNA region it is necessary to modify the amplification temperature profile which is typically practiced. The principal modifications consist of the use of substantially longer annealing times, in a range of about 3 to about 7 minutes. Amplification reactions are being run under high stringency conditions in conjunction with a decreased number of amplification cycles. A high stringency amplification is accomplished by running the reaction at the highest annealing temperature where products are reproducibly formed. Use of maximum annealing temperature insures that only the most stable hybridization structures will form and that the areas surrounding the priming sites will possess a minimal amount of secondary structure.

The presence or absence target nucleic acids can be determined, e.g., by analyzing the amplified nucleic acid products of the primer extension by size using standard methods, for example, agarose gel electrophoresis, polyacrylamide gel electrophoresis, capillary electrophoresis, pulsed field electrophoresis, denatured gradient gel electrophoresis, DNA microarrays, or mass spectrometry. Preferably, capillary electrophoresis is used to separate the amplified products.

In capillary electrophoresis, the length of a nucleic acid fragment is examined by allowing a sample to migrate through a thin tube filled with gel and measuring a period of time required for the sample to migrate a certain distance (e.g., to the end of a capillary). Upon capillary electrophoresis, it is usual to detect a sample using a fluorescence signal detector that is installed at the end of a capillary.

Apparatuses for carrying out capillary electrophoresis are well-known. Many references are available describing the basic apparatus and several capillary electrophoresis instruments are commercially available, e.g., from Applied Biosystems (Foster City, Calif.). Exemplary references describing capillary electrophoresis apparatus and their operation include Jorgenson, Methods 4:179-90 (1992); Colburn et al., Applied Biosystems Research News, issue 1 (winter 1990); and the like.

With respect to fluorescence measurement, when PCR is performed using primers labeled at their 5′ ends with a fluorophore, the amplified target sequence is labeled with the detectable fluorescent material, and the intensity of fluorescence emitted from the fluorescent material is measured using a fluorescence spectrophotometer. Suitable fluorophores include, but are not limited to, 6-FAM; Alexa fluor 405, 430, 488, 532, 546, 555, 568, 594, 633, 647, or 660; Cyt; Cy3; Cy3.5; Cy5; Cy5.5; Cy7; hydroxycoumarin; methoxycoumarin; aminocoumarin; fluorescein; HEX; R-phycoerythrin; rhodamine Red-X; ROX; Red 613; Texas Red; allophycocyanin; TruRed; BODIPY 630/650; BODIPY 650/665; BODIPY-FL; BODIPY-R6G; BODIPY-TMR; BODIPY-TRX; carboxyfluorescein; Cascade Blue; 6-JOE; Lissamine rhodamine B; Oregon Green 488, 500, or 514; Pacific Blue; REG; Rhodamine Green; SpectrumAqua; TAMRA; TET; and Tetramethylrhodamine.

As discussed above, preferred primers are disclosed in Table 1. One of the primers in a set or mixture may be labelled with a fluorophore. When the forward primer is labelled with a fluorophore, the reverse primer is not labelled and when the reverse primer is labelled with a fluorophore, the forward primer is not labelled. One embodiment related thereto is for an isolated polynucleotide selected from the group consisting of SEQ ID NO:1, SEQ ID NO:2, SEQ ID NO:3, SEQ ID NO:4, and SEQ ID NO:5, wherein SEQ ID NOs 1 and 5 are labelled with a fluorophore. Another embodiment is for a kit comprising a set of primers comprising PCR primers SEQ ID NO:1 labeled with a fluorophore and at least one of SEQ ID NO:2 and SEQ ID NO:3. In some aspects, the kit comprises both PCR primers SEQ ID NOs: 2 and 3. A further embodiment is for a kit comprising a set of primers comprising PCR primers SEQ ID NO:4 and SEQ ID NO:5, wherein SEQ ID NO:5 is labeled with a fluorophore.

Such a kit may comprise a carrier being compartmentalized to receive in close confinement therein one or more container means, such as tubes or vials. One of said container means may contain unlabeled or detectably-labeled primers. The primers may be present in lyophilized form or in an appropriate buffer as necessary. One or more container means may contain one or more enzymes or reagents to be utilized in PCR reactions. These enzymes may be present by themselves or in admixtures, in lyophilized form or in appropriate buffers. The kit may also contain some or all the additional elements necessary to carry out the PCR and/or CE, such as buffers, extraction reagents, enzymes, pipettes, plates, nucleic acids, nucleoside triphosphates, filter paper, gel materials, transfer materials, autoradiography supplies, and the like.

### General Methods

The following examples are provided to demonstrate preferred embodiments. It should be appreciated by those of skill in the art that the techniques disclosed in the examples which follow represent techniques discovered by the inventor to function well in the practice of the methods disclosed herein, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the presently disclosed methods.

The following abbreviations in the specification correspond to units of measure, techniques, properties, or compounds as follows: “sec” or “s” means second(s), “min” means minute(s), “h” or “hr” means hour(s), “μL” means microliter(s), “mL” means milliliter(s), “L” means liter(s), “μM” means micromolar, “M” means molar, “pmol” means picomole(s), “g” means gram(s), “μg” means microgram(s), “ng” means nanogram(s), “pg” means picogram(s), “CE” means capillary electrophoresis, “bp” means basepair(s), “6-FAM” means 6-carboxyfluorescein.

### EXAMPLE 1

The fungal genomic DNA from Penicillium chrysogenum and Saccharomyces cerevisiae was either purchased from ATCC or extracted using BAX® System Yeast & Mold sample preparation method with modification. Briefly, fresh yeast or mold colony was transferred to the BAX® System disrupter tube (D12522119) containing 1 mL lysis reagent, which was composed of the following components: 12 mL of YM lysis buffer (D12522102), 150 μL of protease (D14128336), 0.36 mL of 20% SDS (D14227145), and 250 μL of DNA stabilizer (D12522119). After disruption at the YM Disrupter Genie (D12522123) for 5 min for yeast and 10 min for mold, tube was heated at 37° C. for 30 min and 95° C. for 10 min, then a 300 μL of cell lysate was loaded to Maxwell® (Promega, Madison, Wis.) for the purification of DNA.

To generate the ribosomal/RAPD DNA profile, a mixture of one forward and one reverse primer was used. Table 1 lists the five primers of the invention. The first primer mixture comprised SEQ ID NOs 1 and 2. The forward primer SEQ ID NO:1 was labeled with 6-FAM dye and contained 11 bp sequence from 185 rDNA plus two extra nucleotides (TG) added at the 5′ end ((6-FAM)-SEQ ID NO:1). The TG sequence does not match the known conserved 18S ribosomal sequence and serves only to make the fluorescence labeled primer a better substrate for the DNA polymerase. To amplify both ribosomal and RAPD fragments, sequence of reverse primer was obtained from a single location of 5.8S rDNA. The sequence of the reverse primer is SEQ ID NO:2. The second primer mixture uses the same 6-FAM labeled SEQ ID NO:1 as forward primer and SEQ ID NO:3 as the reverse primer. The third primer mixture uses SEQ ID NO:4 as the forward primer and 6-FAM labeled SEQ ID NO:5 ((6-FAM)-SEQ ID NO:5) as the reverse primer.

TABLE 1
Primers

| Location of primers | Sequence 5′-3′ |
|---|---|
| 18S-13mer 6-FAM | (6-FAM)-SEQ ID NO: 1 |
| label | |
| 5.8S-11mer | SEQ ID NO: 2 |
| 28S-11mer | SEQ ID NO: 3 |
| 18S-13mer | SEQ ID NO: 4 |
| 28S-13mer 6-FAM | (6-FAM)-SEQ ID NO: 5 |

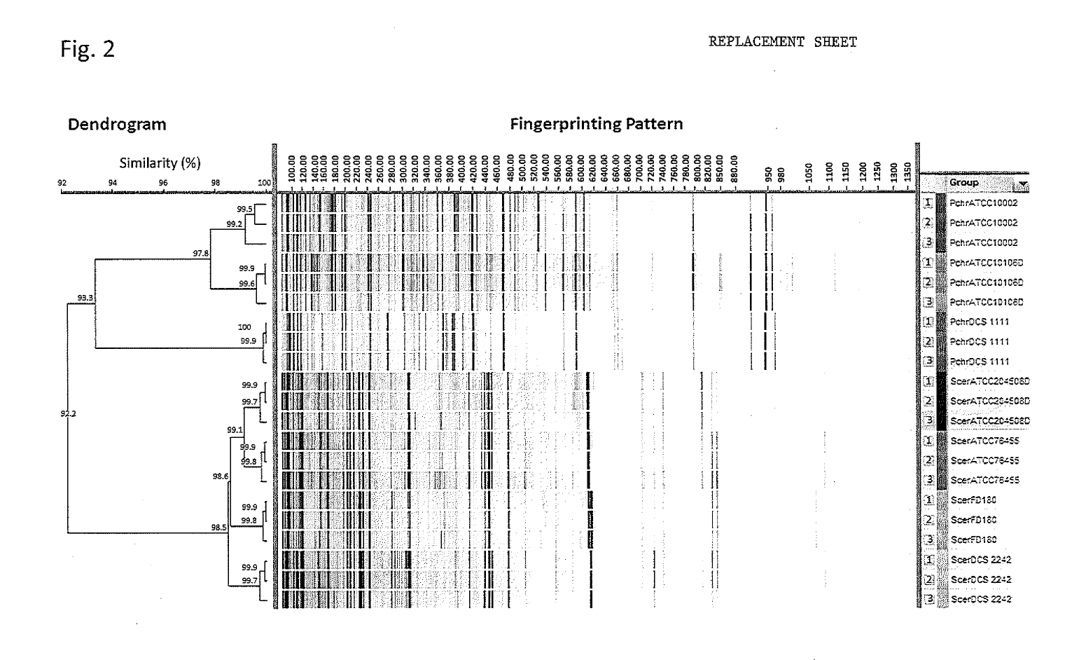

The PCR reaction was performed in 30 μl of reaction mixture, contained 26 μL of BAX® yeast & mold lysis buffer (Part No.:D12522102, 1 μL each of 20 μM of reverse primer, and 2 μL of genomic DNA to rehydrate BAX® primerless tablet (Part No.: D12418797). The PCR fragments were amplified by initial denaturation at 95° C. for 2 min., followed by 40 cycles of 95° C. for 30 sec., 45° C. for 5 min., and 72° C. for 30 sec. The final extension was performed at 72° C. for 10 min. Each DNA sample was run in triplicates during PCR reaction, and the triplicates are labeled as “1”, “2”, “3” in FIG. 2.

A commercial sizing standard (GeneScan 1200LIZ®, Applied Biosystems, Foster City, Calif.) was prepared as follows: 0.5 μl of the size standard was mixed with 9.5 μl of formamide (HiDi, Applied Biosystems). 1.5 μl of the PCR product was then added to the 10 μl size standard/formamide solution. Samples were then mixed and denatured for 2 min at 94° C. then immediately cooled to 4° C., then loaded on to an Applied Biosystems 3730XL Fluorescent Capillary Electrophoresis DNA Sequencer, and run using standard GeneMapper Fragment Analysis Software.

All the capillary electrophoresis data was examined by BioNumerics V7.1 program (Applied Maths Inc, Austin, Tex.) and the cluster analysis was done using Pearson correlation by Unweighted Pair Group Method with Arithmetic mean (UPGMA) method.

Examples of the fingerprinting patterns of the 21 amplification reactions and their cluster hierarchy displayed as dendrogram are shown in FIG. 2. In FIG. 2, both the arbitrary and ribosomal fragments are observed. The dendrogram visually displays three clusters for the three strains of Penicillium chrysogenum and four clusters for the four strains of Saccharomyces cerevisiae. For Penicillium chrysogenum, the similarity between Cluster PchrATCC10002 and Cluster PchrATCC10106D is 97.8%, while the joining of the two clusters makes its similarity with the Cluster PchrDCS1111 at 93.3%. As for the four Saccharomyces cerevisiae clusters, the similarities between Cluster ScerATCC204508D and Cluster ScerATCC76455 is 99.1%, the joining of the two clusters makes its similarity with the Cluster ScerFD180 at 98.6%, and the fusion of the three clusters makes the similarity with Cluster ScerDCS2242 at 98.5%. The joining of the three clusters of Pencillium chrysogenum makes the similarity with the joining of the four clusters of Saccharomyces cerevisiae at 92.2%. The similarity among the triplicates of each individual strain is more than 99.2%.

As demonstrated in FIG. 2, each individual strain of either mold (Penicillium chrysogenum) or yeast (Saccharomyces cerevisiae) was discriminated. The strains with high similarity such as Saccharomyces cerevisiae ATCC204508D and Saccharomyces cerevisiae ATCC76455 in yeast differ in fingerprinting patterns only in the intensities of certain common bands such as 810 bp, 815 bp, 830 bp, and 840 bp etc., while strains of Pencillium chrysogenum ATCC100002 and Pencillium chrysogenum ATCC10106D in mold differ both in the intensities of certain common bands such as 425 bp, 480 bp, 525 bp, 540 bp, and 570 bp etc., and also in the absence of the 845 bp and 850 bp bands in Penicillium chrysogenum ATCC10002.

Similar results were achieved by primer group (6-FAM)-SEQ ID NO:1-SEQ ID NO:3 and SEQ ID NO:4-(6-FAM)-SEQ ID NO:5.

The combination of non-labeled 11-13 bp (SEQ ID NOs: 2, 3, and 4) and 6′FAM-labeled 13 bp primers (SEQ ID NOs: 1 and 5) targeting fungal ribosomal sequences provides amplification of both ribosomal and RAPD fragments under the specified PCR reaction condition. The yields of the ribosomal and dominant RAPD fragments separated by capillary electrophoresis produce a pattern of products that discriminate fungal strain in species and strain level.

### EXAMPLE 2

The fungal genomic DNA from Candida tropicalis, Candida albicans and Aspergillus niger was either purchased from ATCC or extracted using BAX® System Yeast & Mold sample preparation method with modification. Briefly, fresh yeast or mold colony was transferred to the BAX® System disrupter tube (D12522119) containing 1 mL lysis reagent, which was composed of the following components: 12 mL of YM lysis buffer (D12522102), 150 μL of protease (D14128336), 0.36 mL of 20% SDS (D14227145), and 250 μL of DNA stabilizer (D12522119). After disruption at the YM Disrupter Genie (D12522123) for 5 min for yeast and 10 min for mold, tube was heated at 37° C. for 30 min and 95° C. for 10 min, then a 300 μL of cell lysate was loaded to Maxwell® (Promega, Madison, Wis.) for the purification of DNA.

To generate the ribosomal/RAPD DNA profile, a mixture of one forward and one reverse primer is used. The first primer mixture comprises SEQ ID NOs 1 and 2. The forward primer SEQ ID NO:1 is labeled with 6-FAM dye and contained 11 bp sequence from 18S rDNA plus two extra nucleotides (TG) added at the 5′ end ((6-FAM)-SEQ ID NO:1). The TG sequence does not match the known conserved 18S ribosomal sequence and serves only to make the fluorescence labeled primer a better substrate for the DNA polymerase. To amplify both ribosomal and RAPD fragments, sequence of reverse primer is obtained from a single location of 5.8S rDNA. The sequence of the reverse primer is SEQ ID NO:2. The second primer mixture uses the same 6-FAM labeled SEQ ID NO:1 as forward primer and SEQ 1D NO:3 as the reverse primer. The third primer mixture uses SEQ ID NO:4 as the forward primer and 6-FAM labeled SEQ ID NO:5 ((6-FAM)-SEQ ID NO:5) as the reverse primer.

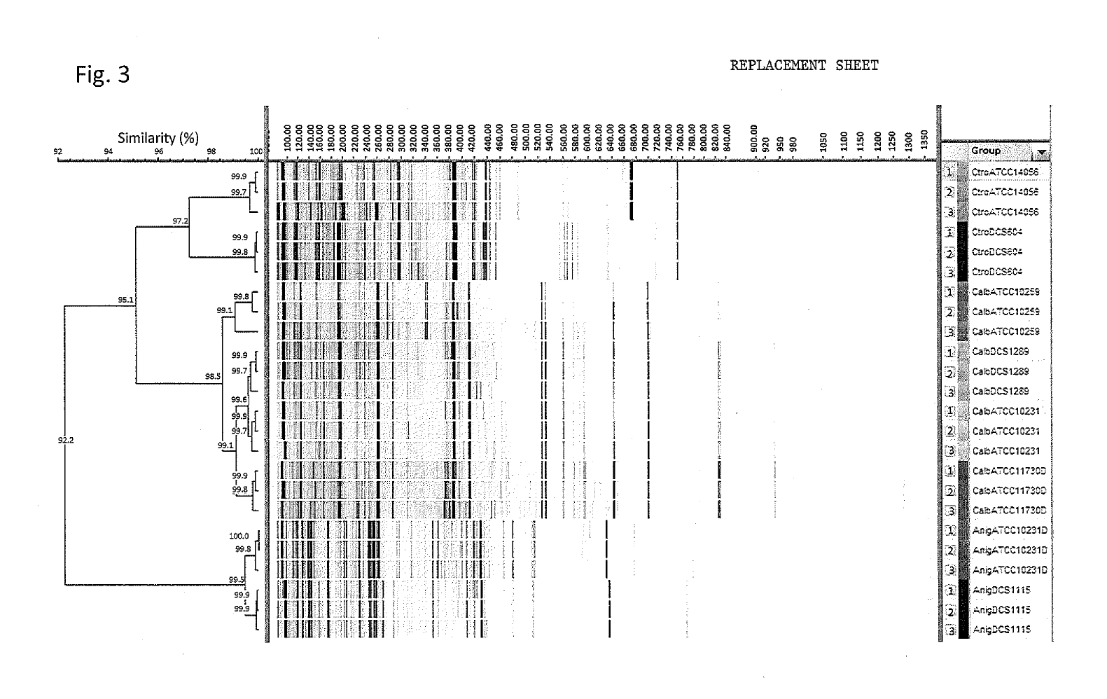

The PCR reaction is performed in 30 μl of reaction mixture, contains 26 μL of BAX® yeast & mold lysis buffer (Part No.: D12522102, 1 μL each of 20 μM of reverse primer, and 2 μL of genomic DNA to rehydrate BAX® primeness tablet (Part No.: D12418797). The PCR fragments are amplified by initial denaturation at 95° C. for 2 min., followed by 40 cycles of 95° C. for 30 sec., 45° C. for 5 min., and 72° C. for 30 sec. The final extension is performed at 72° C. for 10 min. Each DNA sample is run in triplicates during PCR reaction, and the triplicates are labeled as “1”, “2”, “3” in FIG. 3.

A commercial sizing standard (GeneScan 1200LIZ®, Applied Biosystems, Foster City, Calif.) is prepared as follows: 0.5 μl of the size standard is mixed with 9.5 μl of formamide (HiDi, Applied Biosystems). 1.5 μl of the PCR product is then added to the 10 μl size standard/formamide solution. Samples are then mixed and denatured for 2 min at 94° C. then immediately cooled to 4° C., then loaded on to an Applied Biosystems 3730XL Fluorescent Capillary Electrophoresis DNA Sequencer, and run using standard GeneMapper Fragment Analysis Software.

All the capillary electrophoresis data is examined by BioNumerics V7.1 program (Applied Maths Inc, Austin, Tex.) and the cluster analysis is done using Pearson correlation by Unweighted Pair Group Method with Arithmetic mean (UPGMA) method.

Examples of the fingerprinting patterns of the 24 amplification reactions and their cluster hierarchy displayed as dendrogram are shown in FIG. 3. In FIG. 3, both the arbitrary and ribosomal fragments are observed. The dendrogram visually displays two clusters for the two strains of Candida tropicalis, four clusters for the four strains of Candida albicans, and two clusters for the two strains of Aspergillus niger. For Candida tropicalis, the similarity between Cluster CtroATCC14056 and Cluster CtroDCS604 is 97.2%. As for the four Candida albicans clusters, the similarities between Cluster CalbDCS1289 and Cluster CalbATCC10231 is 99.6%, the joining of the two clusters makes its similarity with the Cluster CalbATCC117300 at 99.1%, and the fusion of the three clusters makes the similarity with Cluster CalbATCC10259 at 98.5%. For the two Aspergillus niger, the similarity between Cluster AnigATCC10231D and Cluster AnigDCS1115 is 99.5%. The joining of the two clusters of Candida tropicalis makes the similarity with the joining of the four clusters of Ccandida albicans at 95.1%, and the fusion of the clusters of the two Candida species makes the similarity with the cluster of Aspergillus niger at 92.2%. The similarity among the triplicates of each individual strain is more than 99.1%.

As demonstrated in FIG. 3, each individual strain of yeast (Candida albicans or Candida tropicalis) or mold (Aspergillus niger) was discriminated. The strains with high similarity such as Candida albicans DCS1289, Candida albicans ATCC10231 and Candida albicans ATCC11730D in yeast differ in fingerprinting patterns only in the intensities of certain common bands such as 100 bp, 140 bp, and 165 bp etc., while they are different from Candida albicans ATCC10259 in the absence of the 275 bp, 340 bp and 345 bp bands in Candida albicans DCS1289, Candida albicans ATCC10231 and Candida albicans ATCC11730D.

Similar results were achieved by primer group (6-FAM)-SEQ ID NO:1-SEQ ID NO:3 and SEQ ID NO:4-(6-FAM)-SEQ ID NO:5.

The combination of non-labeled 11-13 bp (SEQ ID NOs: 2, 3, and 4) and 6′FAM-labeled 13 bp primers (SEQ ID NOs: 1 and 5) targeting fungal ribosomal sequences provides amplification of both ribosomal and RAPD fragments under the specified PCR reaction condition. The yields of the ribosomal and dominant RAPD fragments separated by capillary electrophoresis produce a pattern of products that discriminate fungal strain in species and strain level.

### EXAMPLE 3

The fungal genomic DNA from Cryptococcus neoformans, Fusarium solani, Rhodotorula mucilaginosa and Zygosaccharomyces rouxii was either purchased from ATCC or extracted using BAX® System Yeast & Mold sample preparation method with modification. Briefly, fresh yeast or mold colony was transferred to the BAX® System disrupter tube (D12522119) containing 1 mL lysis reagent, which was composed of the following components: 12 mL of YM lysis buffer (D12522102), 150 μL of protease (D14128336), 0.36 mL of 20% SDS (D14227145), and 250 μL of DNA stabilizer (D12522119). After disruption at the YM Disrupter Genie (D12522123) for 5 min for yeast and 10 min for mold, tube was heated at 37° C. for 30 min and 95° C. for 10 min, then a 300 μL of cell lysate was loaded to Maxwell® (Promega, Madison, Wis.) for the purification of DNA.

To generate the ribosomal/RAPD DNA profile, a mixture of one forward and one reverse primer is used. The first primer mixture comprises SEQ ID NOs 1 and 2. The forward primer SEQ ID NO:1 is labeled with 6-FAM dye and contained 11 bp sequence from 18S rDNA plus two extra nucleotides (TG) added at the 5′ end ((6-FAM)-SEQ ID NO:1). The TG sequence does not match the known conserved 18S ribosomal sequence and serves only to make the fluorescence labeled primer a better substrate for the DNA polymerase. To amplify both ribosomal and RAPD fragments, sequence of reverse primer is obtained from a single location of 5.8S rDNA. The sequence of the reverse primer is SEQ ID NO:2. The second primer mixture uses the same 6-FAM labeled SEQ ID NO:1 as forward primer and SEQ ID NO:3 as the reverse primer. The third primer mixture uses SEQ ID NO:4 as the forward primer and 6-FAM labeled SEQ ID NO:5 ((6-FAM)-SEQ ID NO:5) as the reverse primer.

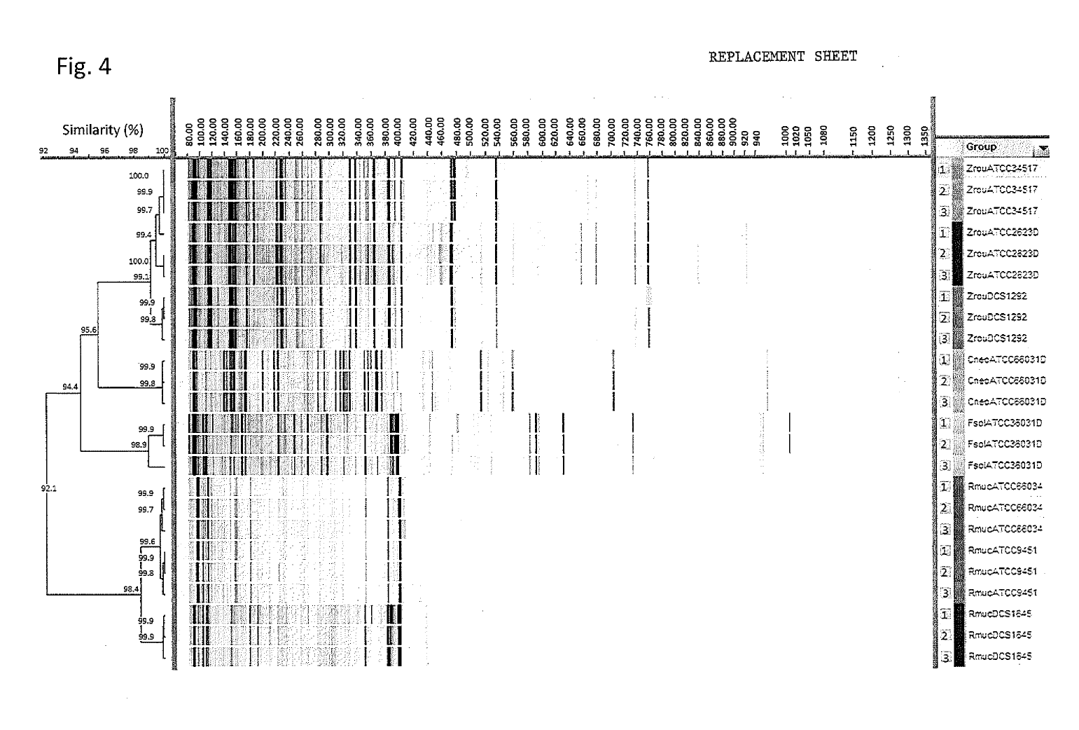

The PCR reaction is performed in 30 μl of reaction mixture, contains 26 μL of BAX® yeast & mold lysis buffer (Part No.: D12522102, 1 μL each of 20 μM of reverse primer, and 2 μL of genomic DNA to rehydrate BAX® primerless tablet (Part No.: D12418797). The PCR fragments are amplified by initial denaturation at 95° C. for 2 min., followed by 40 cycles of 95° C. for 30 sec., 45° C. for 5 min., and 72° C. for 30 sec. The final extension is performed at 72° C. for 10 min. Each DNA sample is run in triplicates during PCR reaction, and the triplicates are labeled as “1”, “2”, “3” in FIG. 4.

A commercial sizing standard (GeneScan 1200LIZ®, Applied Biosystems, Foster City, Calif.) is prepared as follows: 0.5 μl of the size standard is mixed with 9.5 μl of formamide (HiDi, Applied Biosystems). 1.5 μl of the PCR product is then added to the 10 μl size standard/formamide solution. Samples are then mixed and denatured for 2 min at 94° C. then immediately cooled to 4° C., then loaded on to an Applied Biosystems 3730XL Fluorescent Capillary Electrophoresis DNA Sequencer, and run using standard GeneMapper Fragment Analysis Software.

All the capillary electrophoresis data is examined by BioNumerics V7.1 program (Applied Maths Inc, Austin, Tex.) and the cluster analysis is done using Pearson correlation by Unweighted Pair Group Method with Arithmetic mean (UPGMA) method.

Examples of the fingerprinting patterns of the 24 amplification reactions and their cluster hierarchy displayed as dendrogram are shown in FIG. 4. In FIG. 4, both the arbitrary and ribosomal fragments are observed. The dendrogram visually displays three clusters for the three strains of Zygosaccharomyces rouxii, one cluster for the one strain of Cryptococcus neoformans, one cluster for the one strain of Fusarium solani, and three clusters for the three strains of Rhodotorula mucilaginosa. For Zygosaccharomyces rouxii, the similarity between Cluster ZrouATCC34517 and Cluster ZrouATCC2823D is 99.4%, while the joining of the two clusters makes its similarity with the Cluster ZrouDCS1292 at 99.1%. As for the three Rhodotorula mucilaginosa clusters, the similarities between Cluster RmucATCC66034and Cluster RmucATCC9451 is 99.6%, the joining of the two clusters makes its similarity with the Cluster RmucDCS1645 at 98.4%. The joining of the three clusters of Zygosaccharomyces rouxii makes the similarity with the one cluster of Cryptococcus neoformans at 95.6%, while their fusion makes the similarity with the cluster of Fusarium solani at 94.4%, and the joining of the clusters of Zygosaccharomyces rouxii, Cryptococcus neoformans, and Fusarium solani makes the similarity with the joining of the three clusters of Rhodotorula mucilaginosa at 92.1%. The similarity among the triplicates of each individual strain is more than 98.9%.

As demonstrated in FIG. 4, each individual strain of mold (Fusarium solani) or yeast (Cryptococcus neoformans, Rhodotorula mucilaginosa, and Zygosaccharomyces rouxii) was discriminated. The strains with high similarity such as Rhodotorula mucilaginosa ATCC66034, Rhodotorula mucilaginosa ATCC9451 and Rhodotorula mucilaginosa DCS1645 in yeast differ in fingerprinting patterns only in the intensities of certain common bands such as 110 bp, 120 bp, 150 bp, 160 bp, 185 bp, 195 bp and 435 bp etc.

Similar results were achieved by primer group (6-FAM)-SEQ ID NO:1-SEQ ID NO:3 and SEQ ID NO:4-(6-FAM)-SEQ ID NO:5.

The combination of non-labeled 11-13 bp (SEQ ID NOs: 2, 3, and 4) and 6′FAM-labeled 13 bp primers (SEQ ID NOs: 1 and 5) targeting fungal ribosomal sequences provides amplification of both ribosomal and RAPD fragments under the specified PCR reaction condition. The yields of the ribosomal and dominant RAPD fragments separated by capillary electrophoresis produce a pattern of products that discriminate fungal strain in species and strain level.

What is claimed is:

1. A method for the identification of the species, serotype, and strain of a fungi comprising:
a. amplifying DNA comprising variable sequences interspersed between highly conserved rDNA sequences by PCR and amplifying additional genomic sequences by random amplified polymorphic DNA (RAPD) PCR using a first primer of at least 13 bases in length and a second primer of at least 11-13 bases in length, said first primer comprising:
i. at least 11 contiguous bases from a highly conserved 18S rDNA region and an at least 2 base mismatch; and
b. separating the amplified DNA produced in step (a).

2. The method of claim 1, wherein said first primer is a forward primer and said second primer is a reverse primer.

3. The method of claim 1, wherein said first primer further comprises a fluorescent label and said second primer does not include a fluorescent label.

4. The method of claim 2, wherein said first primer is selected from the group consisting of SEQ ID NO:1 labeled with a fluorophore and SEQ ID NO: 4.

5. The method of claim 1, wherein said second primer comprises at least 11-13 contiguous bases from the group consisting of 5.8S rDNA and 28S rDNA.

6. The method of claim 5, wherein said second primer is selected from the group consisting of SEQ ID NO:2, SEQ ID NO:3, and SEQ ID NO:5, wherein SEQ ID NO:5 is labeled with a fluorophore.

7. The method of claim 1 wherein said second primer further comprises a fluorescent label and said first primer does not include a fluorescent label.

8. The method of claim 1 wherein either the first primer or the second primer further comprises a fluorescent label.

9. The method of claim 1, wherein step (b) is accomplished by capillary electrophoresis.

10. An isolated polynucleotide selected from the group consisting of SEQ ID NO:1, SEQ ID NO:2, SEQ ID NO:3, SEQ ID NO:4, SEQ ID NO:5, and the full-length complements thereof.

11. A kit comprising a set of primers comprising at least one of forward PCR primers SEQ ID NO:1 and SEQ ID NO:4, and at least one of reverse PCR primers SEQ ID NO:2, SEQ ID NO:3, and SEQ ID NO:5, wherein SEQ ID NO: 1 and SEQ ID NO:5 are labeled with a fluorophore.

12. A kit comprising a set of primers comprising PCR primer SEQ ID NO:1 and at least one primer selected from the group consisting of SEQ ID NO:2, SEQ ID NO:3, and mixtures thereof, wherein SEQ ID NO:1 is labelled with a fluorophore.

13. A kit comprising a set of primers comprising PCR primers SEQ ID NO:4 and SEQ ID NO:5, wherein SEQ ID NO:5 is labelled with a fluorophore.
